(19) United States
(12) Patent Application Publication
(10) Pub. No.: US 2005/0137586 A1
(43) Pub. Date: Jun. 23, 2005

(54) Hybrid eye tracking system and associated methods

(76) Inventors: Gary P. Gray, Orlando, FL (US); John A. Campin, Orlando, FL (US); Steven E. Bott, Oviedo, FL (US); David M. Gibbs, Orlando, FL (US); George H. Pettit, Maitland, FL (US)

Correspondence Address: ALCON RESEARCH, LTD., R&D COUNSEL, Q-148, 6201 SOUTH FREEWAY, FORT WORTH, TX, 76134-2099, US

(21) Appl. No.: 11/020,429

(22) Filed: Dec. 22, 2004

Related U.S. Application Data
(60) Provisional application No. 60/532,041, filed on Dec. 23, 2003.

(51) Int. Cl. A61B 18/20

(52) U.S. Cl. 606/5; 606/10

(57) ABSTRACT

A system and method for tracking ocular changes during a surgical procedure include directing an eye-safe optical beam toward an undilated, unparalyzed eye. A reflected optical beam is detected, and measurements are performed based upon data contained in the reflected optical beam of at least one geometric parameter of the eye at a predetermined frequency, and from them is calculated a change in the at least one geometric parameter. The calculated change is used to dynamically adjust the directing of laser beam shots during the surgery.

### FIELD OF THE INVENTION

The present invention relates to systems and methods for performing eye tracking, such as during laser surgery, and, more particularly, to such systems and methods for tracking both rapid and slow eye movements.

### DESCRIPTION OF RELATED ART

The measurement of wavefront aberrations emanating from an eye is known in the art to provide data for driving laser-surgical corrective systems. Various embodiments of a method and system for objectively measuring and surgically correcting aberrations of optical systems by wavefront analysis have been disclosed in commonly owned application Ser. No. 09/566,668, “Apparatus and Method for Objective Measurement and Correction of Optical Systems Using Wavefront Analysis,” filed May 8, 2000, and in commonly owned issued U.S. Pat. Nos. 5,632,742 and 5,980,513, all of which are hereby incorporated by reference herein.

Corrective laser surgery may be performed, for example, by laser ablation of portions of the corneal surface to achieve a calculated shape for improving visual acuity. In this case it is desirable to account for eye movement during surgery while delivering laser shots to the cornea. Rapid, involuntary (“saccadic”) eye movement comprises motion of very short duration, in the 10-20 msec range, and up to 1□ of rotation. This movement makes it difficult to determine a visual axis from which to calculate a movement of a treatment laser. The '513 patent addresses this problem with an x,y eye tracking system such as will be described more fully in the following.

Two other types of eye movement, which are slower than saccadic eye movement, comprise cyclo-torsion and translation movements. Translation movement is at present eliminated by paralyzing the pupil so that asymmetric pupil dilation and constriction do not shift the tracking point, resulting in decentration. Manual observation of indicia such as ink marks and/or reticle adjustment are typically used to account for cyclo-torsion during the procedure, which can result from postural changes between the patient's measurement and surgical positions.

### SUMMARY OF THE INVENTION

It is therefore an object of the present invention to provide a system and method for tracking eye movement during laser surgery to correct ocular aberrations.

It is a further object to provide a system and method for tracking eye movement that obviates the need for eye dilation during laser surgery.

It is another object to provide a system and method for tracking eye movement that obviates the need for paralyzing the patient's pupil during laser surgery.

It is also an object to provide a system and method for tracking both rapid and slow eye movement.

It is an additional object to provide a system and method for automating adjustments to beam delivery parameters during laser surgery.

These and other objects are achieved by the present invention, a hybrid eye tracking system and associated methods. The method is for tracking ocular changes during a surgical procedure such as laser vision corrective surgery, and comprises the step of directing an eye-safe optical beam toward an eye. The eye can be substantially untreated (minimally treated by drugs or otherwise) to achieve dilation and paralysis. Next a reflected optical beam from the eye is detected, and a plurality of measurements are performed based upon data contained in the reflected optical beam. The measurements are made of at least one geometric parameter of the eye at a predetermined frequency, and from them is calculated a change in the at least one geometric parameter.

Another aspect of the invention is directed to a method for performing a corrective procedure upon an eye. This method comprises the steps of directing a plurality of ablating laser beam shots at a cornea of an eye in a predetermined pattern. Again, the eye can be substantially untreated to achieve dilation and paralysis. Ocular changes are tracked as above. Ocular changes are compensated for by dynamically adjusting the directing of the laser beam shots based upon the calculated change.

Systems are also provided as part of the present invention for performing the above-described methods.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWINGS

A more complete understanding of the present invention and the advantages thereof may be acquired by referring to the following description, taken in conjunction with the accompanying drawings in which like reference numbers indicate like features and wherein:

FIG. 1 is a schematic diagram of the hybrid eye tracking system of the present invention;

FIG. 2 is a flow diagram of the slow movement tracker control system of the present invention; and

FIG. 3 is a schematic diagram of an eye, illustrating the parameters monitored by the system.

### DETAILED DESCRIPTION OF THE PREFERRED EMBODIMENTS

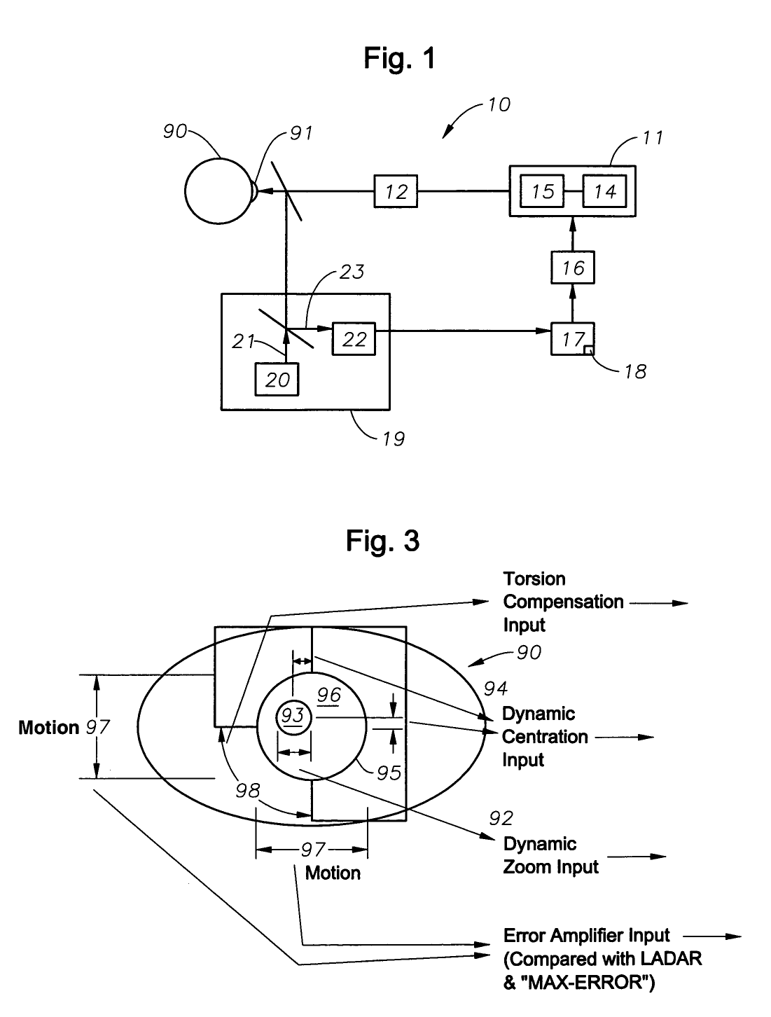

A description of the preferred embodiments of the present invention will now be presented with reference to FIGS. 1-3.

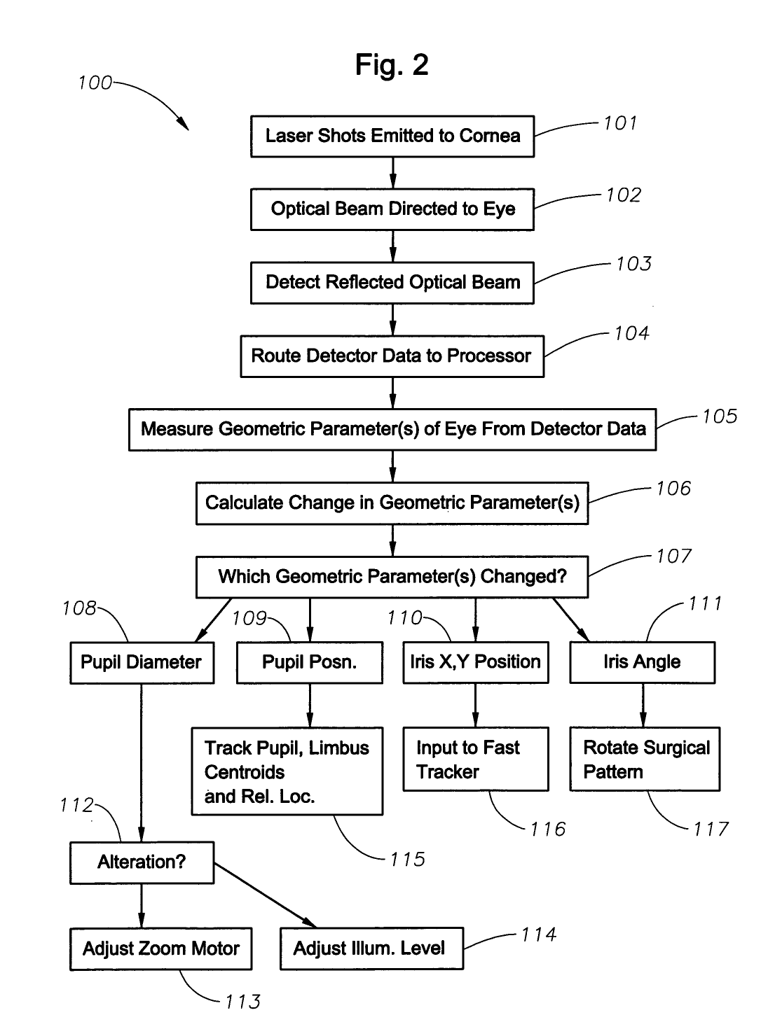

A system 10 (FIG. 1) and method 100 (FIG. 2) for performing a corrective procedure upon an undilated, unparalyzed eye are provided. The system 10 comprises a surgical component 11 and a tracking component, which in turn comprises a rapid eye movement (saccadic) tracker 12, such as disclosed in the '513 patent, and a slow movement tracker 13. The tracking components 12,13 are for tracking ocular changes during the surgical procedure.

The surgical component 11 comprises an ablating laser 14 and associated optics 15 adapted for emitting laser beam shots at a cornea 91 of an eye 90 in a predetermined pattern (block 101) based upon, for example, a wavefront measurement, although this is not intended as a limitation. The surgical component 11 is under control of a beam translation component 16 that is in turn under control of a processor 17 having software 18 resident thereon. The software 18 comprises means for performing calculations on data received from the tracking components 12,13.

The slow tracker 13 comprises a video system 19 comprising an illumination means 20 adapted to direct an eye-safe optical beam 21 toward the eye 90 (block 102). The video system 19 further comprises a detector 22 for detecting a reflected optical beam 23 from the eye 90 (block 103), tracking features such as, for example, scleral features such as blood vessels, limbus shape and ellipticity, iris features, and artificial eye marks. Data from the detector 22 are routed to the processor 17 (block 104), from which a plurality of measurements are made of at least one geometric parameter of the eye 90 at a predetermined frequency (block 105), in one embodiment preferably less than 250 Hz. From these measurements is calculated a change in the at least one geometric parameter (block 106), including a translational and/or a cyclo-torsional change.

The geometric parameter (see FIG. 3) may comprise (block 107), for example, at least one of a diameter 92 of a pupil 93 (block 108), a pupil (centroid) position 94 relative to the limbus 95 (block 109), and a position of the iris 96 in x,y coordinates 97 (block 110) and as a cyclo-torsion angle 98 (block 111).

If the pupil diameter 92 changes, either (block 112) the zoom motor 24 adjusts (block 113) or the ambient illumination level in the room is adjusted to maintain a substantially constant pupil diameter 92 (block 114). Preferably this measurement is updated at a rate approximately 10 times the rate of change of the pupil diameter 92.

If the pupil position 94 changes relative to the limbus 95, an x,y offset is calculated from the limbus 95, and the centroids of the pupil 93 and the limbus 95 are tracked, along with their relative locations (block 115). Preferably this measurement is updated at a rate approximately 10 times the rate of change of the pupil diameter 92. Pupil centroid offset or “pupil drift” depends on the state of the iris and may be larger or smaller from patient to patient and may increase or decrease slightly during surgery. This displacement may not be noticed by the fast iris/pupil boundary tracker and may need to be added vectorally to every shot position with the excimer scanner mirrors so that the shots end up on the desired locations on the cornea independent of pupil centroid drift.

A preferred way to account for pupil center drift due to pupil size changes is to characterize the pupil center position relative to the center of the limbus at the pre-operative visit using the video images of the pupil under different lighting conditions. An equation for how pupil center shifts with respect to size changes of the pupil can be developed from such a pre-operative evaluation. It is assumed that during surgery, if the video tracker observed pupil size changes, the same drift offset will be present. This means that an equal and opposite offset in the location of where the excimer scanners will place these shots must be applied to “null out” the effect that the pupil drift would have on the high speed tracked position, which is only closed around the real-time pupil center and will not account for changes in relative position of the pupil center with respect to the limbus or cornea.

If the iris position in x,y coordinates 97 changes relative to the excimer laser center, due to, for example, the normal head shifts, chin rolling or other movements that cause the entire eye to move, the eye will be tracked-in using the high-speed four IR spot closed-loop LADAR tracker, which uses the tracking galvonometer mirrors to constantly adjust the position of the tracked eye to maintain a space stabilized image

If the rotational position 98 of the iris 96 changes, the new position is used to calculate and perform a rotation of the surgical pattern generated by the excimer scanning mirrors to compensate for cyclo-torsion (block 117). Video tracking may be performed using iris feature recognition, scleral blood vessel detection 96 or on features marked on the eye 90. Preferably this measurement is updated at a rate 10 times the rate of change of the ocular rotation.

Ocular changes noted on the video images that cause the locations on the cornea of the desired laser ablation pattern to be shifted relative the location of the excimer beam scanning positions are compensated for by dynamically adjusting the directing of the laser beam shots via the excimer laser scanning mirrors based upon the calculated change.

It will be seen by one of skill in the art that other embodiments and uses may be contemplated for the present invention. For example, the eye tracker monitoring system and method may be used in settings other than surgical sites, including such sites as for psychological and physiological testing applications.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

Having now described the invention, the construction, the operation and use of preferred embodiment thereof, and the advantageous new and useful results obtained thereby, the new and useful constructions, and reasonable mechanical equivalents thereof obvious to those skilled in the art, are set forth in the appended claims.

1. A method of tracking ocular changes during a surgical procedure, the method comprising the steps of:
directing an eye-safe optical beam toward an eye, the eye substantially untreated to achieve dilation and paralysis;
detecting a reflected optical beam from the eye;
performing a plurality of measurements based upon data contained in the reflected optical beam of at least one geometric parameter of the eye at a predetermined frequency; and
calculating from the measurements a change in the at least one geometric parameter.

2. The method recited in claim 1, wherein the geometric parameter comprises at least one of a pupil diameter, a pupil position relative to a limbus of the eye, and an iris position.

3. The method recited in claim 1, wherein the optical beam comprises a Class 1 Laser.

4. The method recited in claim 1, wherein the detecting step comprises performing video monitoring.

5. The method recited in claim 4, wherein the video monitoring step is performed at a detection rate less than 250 Hz.

6. The method recited in claim 1, wherein the calculating step comprises determining at least one of a cyclo-torsion and a translation of an eye component.

7. A method of performing a corrective procedure upon an eye comprising the steps of:
directing a plurality of ablating laser beam shots at a cornea of an eye in a predetermined pattern, the eye substantially untreated to achieve dilation and paralysis;
tracking ocular changes by:
directing an eye-safe optical beam toward an eye;
detecting a reflected optical beam from the eye;
performing a plurality of measurements based upon data contained in the reflected optical beam of at least one geometric parameter of the eye at a predetermined frequency; and
calculating from the measurements a change in the at least one geometric parameter; and
compensating for ocular changes by dynamically adjusting the directing of the laser beam shots based upon the calculated change.

8. The method recited in claim 7, wherein the detecting step comprises performing video monitoring.

9. The method recited in claim 7, wherein the video monitoring step is performed at a detection rate less than 250 Hz.

10. The method recited in claim 7, wherein the calculating step comprises determining at least one of a cyclo-torsion and a translation of an eye component.

11. The method recited in claim 7, wherein the at least one geometric parameter comprises a rotation of an iris of the eye.

12. The method recited in claim 11, wherein the adjusting step comprises rotating the predetermined pattern to compensate for the iris rotation.

13. The method recited in claim 7, wherein the at least one geometric parameter comprises a position of a pupil of the eye relative to an iris of the eye.

14. The method recited in claim 13, wherein the adjusting step comprises translating the predetermined pattern to compensate for the pupillary position.

15. The method recited in claim 7, further comprising the step of tracking saccadic eye movement using a boundary between the pupil and an iris of the eye.

16. The method recited in claim 15, wherein the at least one geometric parameter comprises a diameter of a pupil of the eye.

17. The method recited in claim 16, wherein the adjusting step comprises providing input for the saccadic eye movement tracking.

18. The method recited in claim 16, further comprising the step of adjusting a level of illumination surrounding the eye to maintain a substantially constant pupil diameter.

19. The method recited in claim 15, wherein the at least one geometric parameter comprises a position of a limbus of the eye.

20. The method recited in claim 19, wherein the adjusting step comprises providing input for the saccadic eye movement tracking.

21. A system for tracking ocular changes during a surgical procedure comprising:
means for directing an eye-safe optical beam toward an eye, the eye substantially untreated to achieve dilation and paralysis;
a video detector for collecting reflected optical beam data from the eye at a predetermined frequency; and
software means installable on a processor for calculating from the collected data a change in at least one geometric parameter of the eye.

22. The system recited in claim 21, wherein the geometric parameter comprises at least one of a pupil diameter, a pupil position relative to a limbus of the eye, and an iris position.

23. The system recited in claim 21, wherein the optical beam comprises a Class 1 Laser.

24. The system recited in claim 21, wherein the detector comprises performing video monitor.

25. The system recited in claim 24, wherein the video monitor is adapted to collect data at a detection rate less than 250 Hz.

26. The system recited in claim 21, wherein the software means is adapted to determine at least one of a cyclo-torsion and a translation of an eye component.

27. A system for performing a corrective procedure upon an eye comprising:
means for directing a plurality of ablating laser beam shots at a cornea of an eye in a predetermined pattern, the eye substantially untreated to achieve dilation and paralysis;
means for tracking ocular changes comprising:
means for directing an eye-safe optical beam toward an eye;
a video detector for collecting data from a reflected optical beam from the eye at a predetermined frequency;
software means installable on a processor for calculating from the collected data a change in at least one geometric parameter of the eye; and
means for compensating for ocular changes by dynamically adjusting the directing of the laser beam shots based upon the calculated change.

28. The system recited in claim 27, wherein the detector comprises a video monitor.

29. The system recited in claim 27, wherein the video monitor is adapted to collect data at a detection rate less than 250 Hz.

30. The system recited in claim 27, wherein the software means is adapted to determine at least one of a cyclo-torsion and a translation of an eye component.

31. The system recited in claim 27, wherein the at least one geometric parameter comprises a rotation of an iris of the eye.

32. The system recited in claim 31, wherein the adjusting means comprises means for rotating the predetermined pattern to compensate for the iris rotation.

33. The system recited in claim 27, wherein the at least one geometric parameter comprises a position of a pupil of the eye relative to an iris of the eye.

34. The system recited in claim 33, wherein the adjusting means comprises means for translating the predetermined pattern to compensate for the pupillary position.

35. The system recited in claim 27, further comprising means for tracking saccadic eye movement using a boundary between the pupil and an iris of the eye

36. The system recited in claim 35, wherein the at least one geometric parameter comprises a diameter of a pupil of the eye.

37. The system recited in claim 36, wherein the adjusting means comprises means for providing input for the saccadic eye movement tracking.

38. The system recited in claim 36, further comprising means for adjusting a level of illumination surrounding the eye to maintain a substantially constant pupil diameter.

39. The system recited in claim 35, wherein the at least one geometric parameter comprises a position of a limbus of the eye.

40. The system recited in claim 39, wherein the adjusting means comprises means for providing input for the saccadic eye movement tracking.

41. A method of tracking ocular changes during a surgical procedure, the method comprising the steps of:
directing an eye-safe optical beam toward an eye, the eye substantially untreated to achieve dilation and paralysis;
detecting a reflected optical beam from the eye;
performing a plurality of measurements based upon data contained in the reflected optical beam of at least one geometric parameter of the eye at a predetermined frequency;
calculating from the measurements a change in the at least one geometric parameter;
tracking saccadic eye movements; and
using the calculated change in the at least one geometric parameter as input for adjusting the saccadic eye movement tracking.

42. A system for tracking ocular changes during a surgical procedure comprising:
means for directing an eye-safe optical beam toward an eye, the eye substantially untreated to achieve dilation and paralysis;
a video detector for collecting reflected optical beam data from the eye at a predetermined frequency;
a saccadic eye movement tracker; and
software means installable on a processor capable of calculating from the collected data a change in at least one geometric parameter of the eye and using the calculated change in at least one geometric parameter of the eye as input for adjusting the saccadic eye movement tracker.

43. The system recited in claim 42, wherein the saccadic eye movement tracker is a laser-based eye movement tracker.
